(19) United States
(12) Patent Application Publication
(10) Pub. No.: US 2003/0032997 A1
(43) Pub. Date: Feb. 13, 2003

(54) Low impedance high strength medical electrical lead

(76) Inventors: Anne M. Pianca, Valencia, CA (US); James R. Thacker, Eureka, MO (US)

Correspondence Address: ADVANCED BIONICS CORPORATION, 12740 SAN FERNANDO ROAD, SYLMAR, CA, 91342, US

(21) Appl. No.: 10/188,424

(22) Filed: Jul. 2, 2002

Related U.S. Application Data
(60) Provisional application No. 60/311,421, filed on Aug. 10, 2001.

(51) Int. Cl. A61N 1/05

(52) U.S. Cl. 607/117

(57) ABSTRACT

An implantable medical electrical lead provides high strength and low resistance, and is non-toxic and non-neurotoxic. The lead is manufactured from wire combining a high strength material and a low resistance material. Both materials are biocompatible, non-toxic, and in particular, non-neurotoxic. In one embodiment, the wire is a Drawn Filled Tube (DFT) wire, with MP35N® forming a high strength outer shell, and platinum forming a low resistance inner core.

The present application claims the benefit of U.S. Provisional Application Serial No. 60/311,421, filed Aug. 10, 2001, which application is incorporated herein by reference.

### BACKGROUND OF THE INVENTION

The present invention relates to implantable electrical stimulation systems and more particularly to a low impedance, high strength conductor for use with Spinal Cord Stimulation (SCS) systems, Deep Brain Stimulation systems, heart stimulation systems, and other implantable medical devices. Spinal cord stimulation systems treat chronic pain by providing electrical stimulation pulses through the electrodes of an electrode array placed epidurally near a patient's spine. Stimulation current is provided to the electrode array by an Implantable Pulse Generator (IPG). The IPG is connected to the electrode array by a lead, which lead includes conductors to carry the stimulation current.

Implantable electronic medical devices and systems have been in use for the past 20 years or more. One of the earliest implantable medical devices to be implanted in a patient was the cardiac pacemaker. Other implantable electronic devices have included neurostimulators, i.e., electrical stimulators designed to stimulate nerves or other tissue, sensors for sensing various physiological parameters or physical status of a patient, and therapeutic delivery devices, e.g., pumps for delivering controlled amounts of medication. In more recent years, a small implantable cochlear stimulator has been developed that allows patients who are profoundly deaf to experience the sensation of hearing. Other small implantable sensors and neuro-stimulators are under development that will somewhat restore the ability of a patient, who is a recipient of such sensors or stimulators, to walk, or to see, or to experience the use of other lost or impaired body functions.

Most of the implantable medical devices and systems described above require that at least one electrical lead be connected thereto in order for the device or system to perform its intended function. Such lead typically includes a plurality of insulated conductors, or wires, through which electrical signals may be delivered or sensed. An SCS system, for example, typically includes a lead with an electrode array at a distal end of the lead. The electrode array is adapted for insertion into the spinal column of the patient. Such electrode array typically employs a multiplicity of electrode contacts, each of which may be individually electrically connected to the pulse generator circuitry housed within an IPG. The electrode lead associated with such spinal cord stimulator thus carries the individual conductors that electrically connect the respective electrodes to the IPG.

Medical electrical leads which are implantable into the body must be able to survive the harsh environment of the body for an indefinite duration. The body environment can be chemically, electrically, and/or mechanically harsh on an implantable medical electrical lead. The environment may be especially harsh for a lead implanted into the heart or spinal column. A lead implanted into the heart must withstand the fatigue created by the continuous motion of the beating heart, and the pressure generated when forced into contact with a bone. A lead implanted into the spinal column must survive the motion of the spine and torso, and the pressure generated when inserted between vertebrae.

In addition to mechanical strength, an implanted medical electrical lead must be resistance to corrosion, minimally toxic to body tissues, and provide long term electrical stability. For example, it is not desirable to use exposed silver in a lead placed in the nervous system since studies have consistently shown silver to be toxic and necrotic to neural tissue.

In known stimulation systems, a battery residing within the IPG provides the current used for stimulation, which battery contains a limited amount of energy. When the battery is depleted, the stimulation system is rendered useless and the system, or at least the battery, must be explanted and replaced. Due to the inherent cost and risk associated with surgery, it is desirable to minimize the frequency of replacement surgeries a patient must experience, thus it is desirable to maximize the life of the battery.

When selecting biocompatible conductor materials, there is a trade-off between mechanical strength and electrical resistivity. Materials that have excellent long-term mechanical properties, such as MP35N® (an alloy comprising cobalt, nickel, chromium, and molybdenum), typically also have high values of electrical resistivity, and as a result, shorten battery life. In many cases, the impedance measured across electrodes is so high (greater than 200 ohms) that materials such as MP35N®, and the like, are not used for conductors because the life of the device would be too short (e.g., less than 3 years). Some manufacturers have opted to increase the battery size as a method of increasing the life of the device. However, these devices may become so large that they can only be implanted in a limited number of locations in the body, and are often unsightly and uncomfortable for the patient.

In some pacemaker and defibrillation leads, alternative configurations of wire are used to yield conductors that are relatively high in strength, yet low in impedance. Such wire configurations include Drawn Brazed Strand (DBS), and Drawn Filled Tube (DFT). Known DBS wire comprises silver and MP35N® wires that are heated to form a high strength, low resistance composite. DFT wire consists of a solid silver core surrounded by a shell of MP35N®. However, neither of these wire configurations in the forms mentioned are desirable for a lead that is placed in neural tissue because silver is neurotoxic.

What is needed is a wire with high strength and low resistance, that is both non-toxic and in particular non-neurotoxic.

### SUMMARY OF THE INVENTION

The present invention addresses the above and other needs by providing a high strength and low resistance implantable medical electrical lead that is non-toxic and in particular, non-neurotoxic. The lead is manufactured from wire combining a high strength material and a low resistance material. Both materials are biocompatible and non-neurotoxic. In a preferred embodiment, the wire is a Drawn Filled Tube (DFT) wire, with MP35N® forming a high strength outer shell, and platinum forming a low resistance inner core.

In accordance with one aspect of the invention, there is provided a low resistance lead. Stimulation impedance is an important factor to consider in conserving battery energy. The impedance of the stimulation system may be modeled using three major components: the lead conductor resistance (Rₗ), the ohmic resistance of the electrode (Rₑ), and the polarization impedance of the electrode-tissue interface (Zₚₒₗ). The present invention reduces the lead conductor resistance, Rₗ, and thereby extends battery life. When stimulation is delivered at a high duty cycle, as is the case with neurostimulation devices (e.g., Spinal Cord Stimulation (SCS) systems,) reducing system loses becomes even more important than with a device that operates at lower duty cycles such as a pacemaker. In such higher duty cycle devices, the average rate of power consumption is increased, and thus battery life is an important issue. Minimizing the lead impedance advantageously reduces the power required for stimulation and thereby increases battery life.

It is a further feature of the invention to provide a durable lead. Leads implanted into the heart must be resistant to the fatigue created by the continuous motion of the beating heart, and the pressure generated when forced into contact with a bone. Similarly, a lead implanted into the spinal column must endure the motion of the spine and torso, and the pressure generated when inserted between vertebrae. The lead of the present invention includes a high strength material, such as MP35N®, to add strength to the lead.

It is an additional feature to provide a lead that is non-neurotoxic. Known leads include silver to reduce the overall resistance of the lead. While silver may be acceptable in some applications (e.g, pacemakers), it is undesirable where the lead is in contact with tissue of the nervous system. The lead of the present invention replaces silver with a low resistance material which is non-neurotoxic.

It is still another feature of the present invention that the wire can be used with polyurethane insulation. When using a wire which includes silver, if there is any breach in the insulation, saline and protein-rich body fluids can come into direct contact with exposed silver. The chloride ion in solution forms silver chloride on the surface of the exposed silver, resulting in soluble silver complexes. These silver complexes catalyze an oxidation of the ether portion of the polymer molecule. The result is a loss of molecular weight with the polymer becoming weak or brittle. Platinum does not react similarly, and allows the use of polyurethane insolation.

### BRIEF DESCRIPTION OF THE DRAWINGS

The above and other aspects, features and advantages of the present invention will be more apparent from the following more particular description thereof, presented in conjunction with the following drawings wherein:

FIG. 1 shows a Spinal Cord Stimulation (SCS) system including a lead connected to an Implantable Pulse Generator (IPG), and an electrode array;

FIG. 2 depicts the SCS system of FIG. 1 implanted in a spinal column;

FIG. 3 shows details of a lead suitable for use with an SCS system;

FIG. 3A shows a cross-sectional view of the lead taken along line 3A-3A of FIG. 3;

FIG. 4A shows a view of a lead with wires in the form of a cable, with the outer tubing cut away;

FIG. 4B shows a view of a lead with wires in the form of a coil, with the outer tubing cut away;

FIG. 5 depicts a Drawn Filled Tube (DFT) wire according to the present invention; and

FIG. 6 depicts a Drawn Brazed Strand (DBS) wire according to the present invention.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

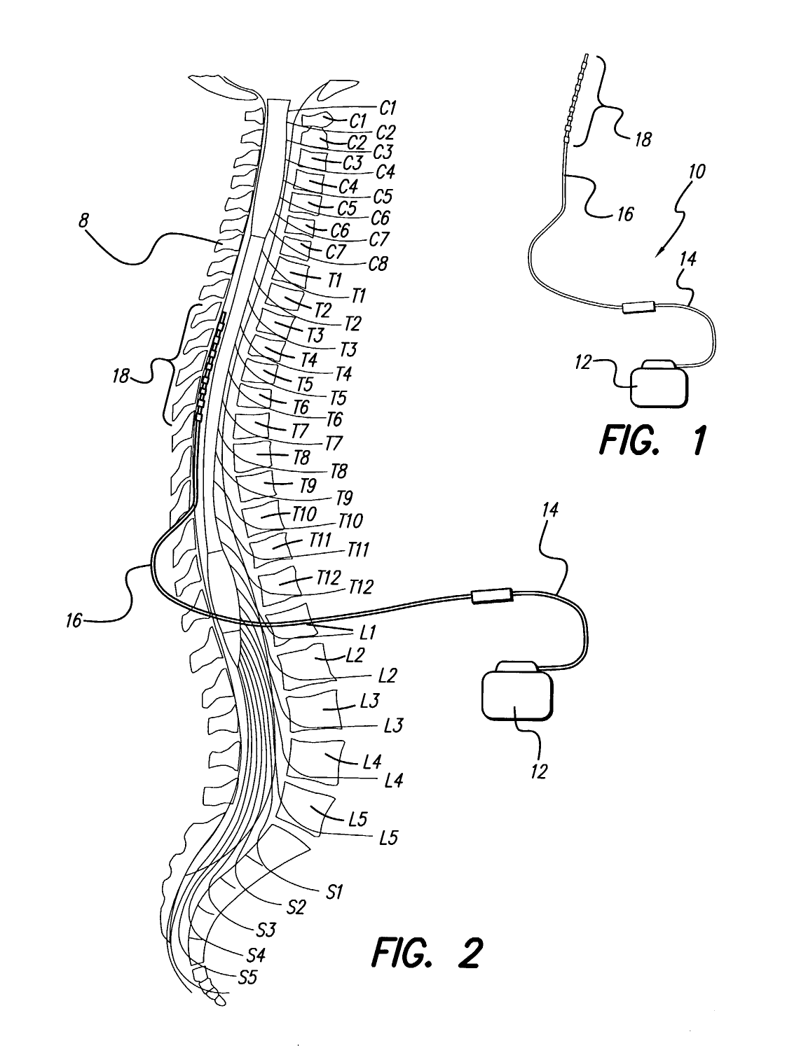

The low impedance high strength medical electrical lead of the present invention provides an improved lead for connecting an Implantable Pulse Generator (IPG) to an electrode array. Such lead is typically used in a Spinal Cord Stimulation (SCS) system 10 as shown in FIG. 1. An SCS system 10 typically comprises an IPG 12, a lead extension 14, and a lead 16 that includes an electrode array 18. The IPG 12 generates stimulation current for implanted electrodes that make up the electrode array 18. A proximal end of the lead extension 14 is removably connected to the IPG 12 and a distal end of the lead extension 14 is removably connected to a proximal end of the lead 16, and the electrode array 18 resides on a distal end of the lead 16. The in-series combination of the lead extension 14 and lead 16, carries the stimulation current from the IPG 12 to the electrode array 18. Both the lead extension 14 and the lead 16 may be constructed according to the lead of the present invention.

The SCS system 10 described in FIG. 1 above, is depicted implanted in a spinal column 8 in FIG. 2. The electrode array 18 is implanted at the site of nerves that are the target of stimulation (e.g., along the spinal cord.) Due to the lack of space near the location where the lead 16 exits the spinal column 8 (the lead exit point), the IPG 12 is generally implanted in the abdomen or above the buttocks. The lead extension 14 facilitates locating the IPG 12 away from the lead exit point.

As seen in FIG. 2, the lead 16 exits the spinal column 8 between vertebrae, and is thereby subjected to pressure and motion. The lead extension 14 tunnels around the waist, and is thereby subjected to motion caused by movement of the patient. The leads 14 and 16 must be sufficiently strong to survive indefinitely in this environment.

While the implantable system depicted in FIGS. 1 and 2 comprises a separate lead extension 14 electrically connecting the lead 16 to the IPG 12, a lead 16 made according to the present invention would apply equally well to a system with a single lead connected between the IPG 12 and the electrode array 18.

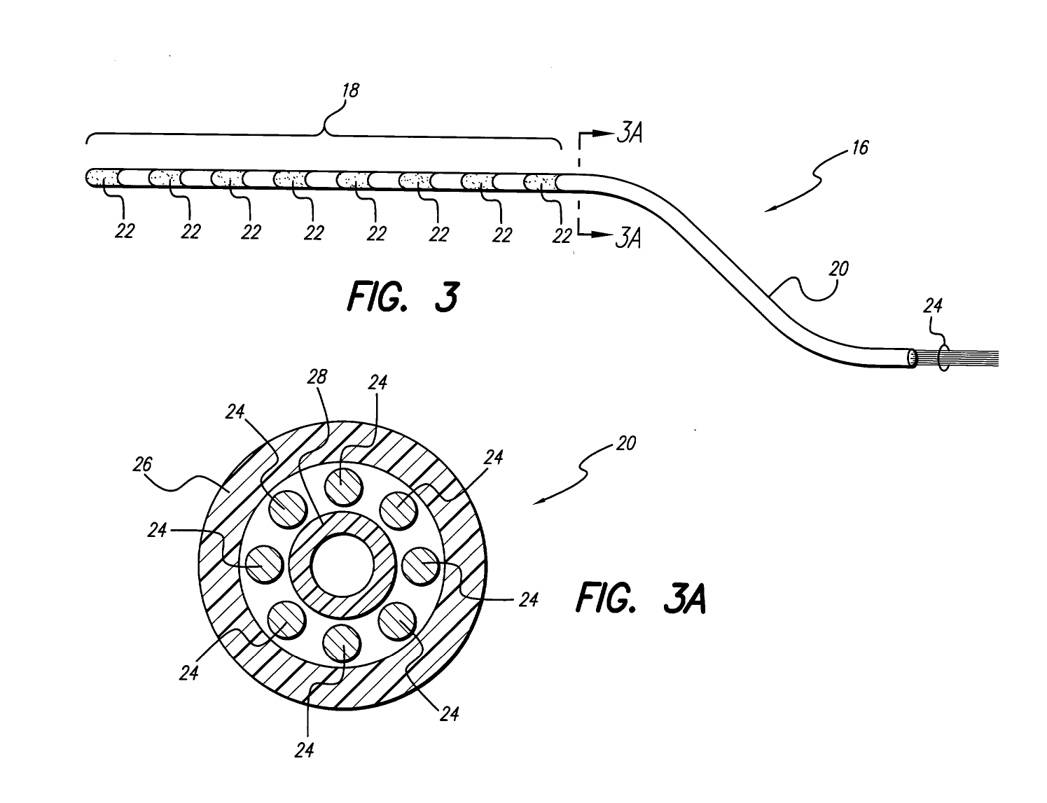

A detailed view of an example of the electrode array 18 end of the lead 16 is shown in FIG. 3. The electrode array 18 comprises a plurality of spaced-apart electrodes 22 along the lead 16 residing at a lead distal end. The lead 16 includes a lead body 20 wherein a plurality of wires 24 reside. Each electrode 22 is electrically connected to at least one of the wires 24. Although the electrode array 18 comprises eight electrodes 22, an electrode array with any number of electrodes may be constructed on a lead according to the present invention, and such lead with differing numbers of electrodes is intended to come within the scope of the present invention.

A cross-sectional view of the lead body 20 taken along line 3A-3A of FIG. 3 is shown in FIG. 3A. The lead body 20 comprises an outer tube 26, an inner tube, or lumen, 28, and a plurality of wires 24 residing between the inner tube 28 and the outer tube 26. The outer tube 26 is preferably made from silicone, or polyurethane, and more preferably from silicone, the inner tube 28 is preferably made from Polytetrafluoroethylene (i.e., Teflon®) (PTFE), Fluorinated Ethylene Propylene (FEP), or polyurethane, and more preferably from PTFE. In other examples, the number of wires 24 may vary, and the inner tube may be absent, or replaced by a solid member.

The lead 16 may also include a connector at a proximal end opposite the distal end. The connector may be a ring contact connector comprising a plurality of spaced apart ring contacts, wherein the connector is similar to the electrode array, but generally shorter with the contacts smaller and closer together than the electrodes. An electrode lead used in a SCS system 10 is preferable between 0.040 and 0.100 inches in diameter.

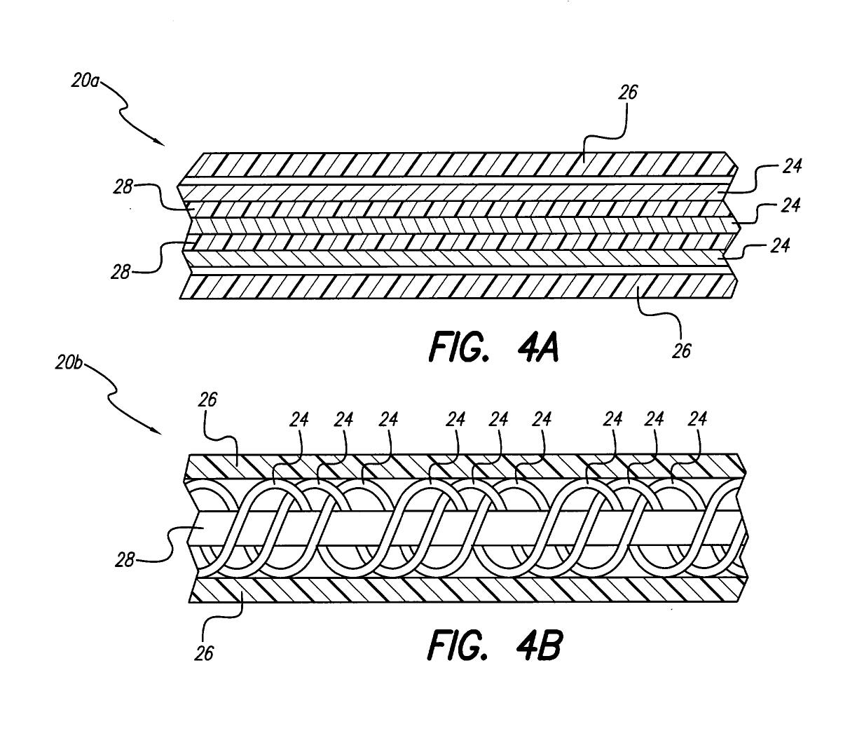

A view of a lead body 20a including wires 24 in the form of a cable is shown in FIG. 4A with the outer tube 26 partially cut away. The wires 24 run parallel to the inner tube 28 in this example.

A view of a lead body 20b including wires 24 in the form of a coil is shown in FIG. 4B with the outer tube 26 partially cut away. The wires 24 are wound around the inner tube 28 in this example.

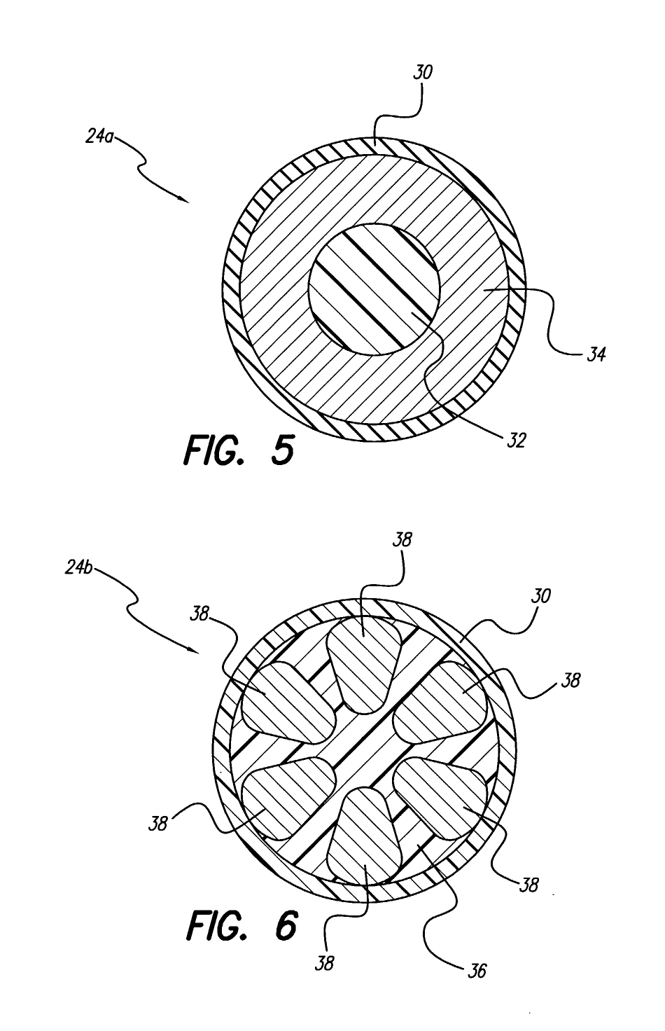

The individual wires used within a lead in accordance with the present invention may take many forms. A preferred wire is a Drawn Filled Tube (DFT) or a Drawn Brazed Strand (DBS). A cross-section of an example of a single DFT wire 24a constructed according to the present invention is shown in FIG. 5. The DFT wire 24a comprises insulation 30, a shell 34 made from a high strength material, and a core 32 made from a low resistance material. The DFT wire may be preferably manufactured by either Fort Wayne Metals in Fort Wayne, Ind., or Nobel-Met in Salem, Va. The insulation 30 is preferably made from Ethylene Tetrafluoroethylene (ETFE), Perfluoroalkoxy (PFA), or PTFE, and more preferably from ETFE. Both the high strength material and the low resistance material are biocompatible, non-toxic, and non-neurotoxic. The high strength material is preferably titanium, tantalum, stainless steel, or MP35N®, and more preferably MP35N® manufactured by the Latrobe Steel Company, Latrobe, Pa. The low resistance material is preferably platinum, tungsten, iridium, gold, or platinum iridium alloy, and more preferably platinum. The low resistance material is preferably about 25% to 45% of the total cross-section of the wire, and more preferably about 28% to 33% of the total cross-section of the wire.

A cross-section of an example of a single DBS wire 24b constructed according to the present invention is shown in FIG. 6. The DBS wire comprises insulation 30, strands 38 made from the high strength material, and a second core 36 made from the low resistance material. The core 36 is surrounded by the strands 38, and the combination is drawn through a die and heated such that the core 36 melts and flows around the strands 38 forming a composite as shown in FIG. 6. The insulation 30, high strength material, and low resistance material are as described above in FIG. 5.

A wire according to the present invention may thus be a DFT wire, a DBS wire, or other type of wire made from a combination of a high strength material and a low resistance material. Any wire constructed comprising a combination of a high strength material, and a low resistance material, wherein both materials are biocompatible, non-toxic, and non-neurotoxic, is intended to come within the scope of the present invention. Further, a lead constructed from wire according to the present invention, is intended to come within the scope of the present invention.

Leads constructed including wires in the form of a cable as shown in FIG. 4A or in the form of a coil as shown in FIG. 4B are known in the art. Such leads are taught in U.S. Pat. No. 6,343,233 issued Jan. 29, 2002 for Medical Lead Adapter,” U.S. Pat. No. 6,216,045 issues Apr. 10, 2001 for “Implantable lead and method of manufacture,” U.S. Pat. No. 5,562,722 issued Oct. 8, 1996 for “Multiple Electrode Catheter,” U.S. Pat. No. 5,423,881 issues Jun. 13, 1995 for “Medical Electrical Lead,” U.S. Pat. No. 5,040,544 issued Aug. 20, 1991 for “Medical Electrical Lead and Method of Manufacture, and” U.S. Pat. No. 4,640,983 issued Feb. 3, 1987 for “Conductor Device, Particularly for at Least Insertion in a Human or Animal Body, Comprising a Spiral Formed From at Least One conductor,” The '233 patent, '045 patent, '722 patent, '881 patent, '544 patent, and '983 patent are incorporated herein by reference.

While the above description describes the application of the present invention to an SCS system, those skilled in the art will recognize that a lead constructed from wire according to the present invention may prove useful in a variety of medical applications. The present invention is not intended to be limited to SCS systems, and the use of wire according to the present invention in these other applications is intended to come within the scope of the present invention.

As described above, the present invention provides a high strength low resistance medical electrical lead which is biocompatible, non-toxic, and non-neurotoxic. By constructing a lead from wire comprising a high strength material and a low resistance material, which materials are both non-toxic and non-neurotoxic, the risks associated with know leads of damaging nerve tissue are avoided. A preferred embodiment comprising a DFT wire formed from MP35N® and platinum was described, along with alternative materials.

While the invention herein disclosed has been described by means of specific embodiments and applications thereof, numerous modifications and variations could be made thereto by those skilled in the art without departing from the scope of the invention set forth in the claims.

What is claimed is:

1. Wire for use in an implantable medical devices, comprising:
a high strength material; and
a low resistance material;
wherein the wire is a Drawn Filled Tube (DFT) wire, and wherein both materials are biocompatible, non-toxic, and non-neurotoxic.

2. The wire of claim 1 wherein the high strength material is selected from a set consisting of titanium, tantalum, stainless steel, and MP35N®.

3. The wire of claim 1 wherein the high strength material is MP35N®.

4. The wire of claim 1 wherein the low resistance material is selected from a set consisting of platinum, tungsten, iridium, gold, and platinum iridium alloy.

5. The wire of claim 1 wherein the low resistance material is platinum.

6. The wire of claim 1 wherein the low resistance material is 25% to 45% of the total cross-section of the wire.

7. The wire of claim 1 wherein the low resistance material is 28% to 33% of the total cross-section of the wire.

8. An implantable medical electrical lead, comprising:
an electrode array comprising a plurality of spaced-apart electrodes residing at a distal end of the lead;
a connector comprising a plurality of spaced-apart contacts, wherein the connector resides at a proximal end of the lead;
a lead body defined between the proximal end and the distal end;
a plurality of wires residing within the lead body, each wire comprising:
a high strength material; and
a low resistance material;
wherein the high strength material and the low resistance material are biocompatible, non-toxic, and non-neurotoxic;
wherein each electrode is electrically connected to at least one of the wires, and wherein each wire is electrically connected to at least one contact.

9. The lead of claim 8 wherein each wire is a Drawn Brazed Strand (DBS) wire.

10. The lead of claim 8 wherein each wire is a Drawn Filled Tube (DFT) wire.

11. The lead of claim 8 wherein the high strength material is selected from a set consisting of titanium, tantalum, stainless steel, and MP35N®.

12. The lead of claim 8 wherein the high strength material is MP35N®.

13. The lead of claim 8 wherein the low resistance material is selected from a set consisting of platinum, tungsten, iridium, gold, and platinum iridium alloy.

14. The lead of claim 8 wherein the low resistance material is platinum.

15. The lead of claim 8 wherein the low resistance material is 25% to 45% of the total cross-section of each wire.

16. The lead of claim 8 wherein the low resistance material is 28% to 33% of the total cross-section of each wire.

17. The lead of claim 8 wherein the lead is constructed from wires in the form of a coil.

18. The lead of claim 8 wherein the lead is constructed from wires in the form of a cable.

19. The lead of claim 8 wherein the lead body includes an outer tube wherein the wires reside within the outer tube.

20. The lead of claim 8 wherein the lead body includes an inner tube wherein the wires reside outside the inner tube.

21. The lead of claim 8 wherein the lead is between 0.040 and 0.100 inches in diameter.

22. A method for constructing an implantable medical electrical lead, comprising:
constructing wire from a high strength material and a low resistance material, wherein the high strength material and the low resistance material are biocompatible, non-toxic, and non-neurotoxic; and
constructing the implantable lead from the wire.

23. The method of claim 22 wherein constructing the wire further comprises constructing a Drawn Filled Tube (DFT) wire.

24. The method of claim 22 wherein constructing the wire further comprises constructing a Drawn Brazed Strand (DBS) wire.

25. The method of claim 22 wherein constructing the wire from a high strength material comprises constructing wire from a high strength material selected from a set consisting of titanium, tantalum, stainless steel, and MP35N®.

26. The method of claim 22 wherein constructing the wire from a high strength material comprises constructing wire from MP35N®.

27. The method of claim 22 wherein constructing the wire from a low resistance material comprises constructing wire from a low resistance material selected from a set consisting of platinum, tungsten, iridium, gold, and platinum iridium alloy.

28. The method of claim 22 wherein constructing the wire from a low resistance material comprises constructing wire from platinum.

29. The method of claim 22 wherein constructing the wire further comprises constructing wire wherein the low resistance material is 25% to 45% of the total cross-section of the wire.

30. The method of claim 22 wherein constructing the wire further comprises constructing wire wherein the low resistance material is 28% to 33% of the total cross-section of the wire.

31. The method of claim 22 wherein constructing a lead from the wire comprises constructing the lead from the wire, wherein the wire in the form of a coil.

32. The method of claim 22 wherein constructing a lead from the wire comprises constructing the lead from the wire, wherein the wire in the form of a cable.
